(19) United States
(12) Patent Application Publication
(10) Pub. No.: US 2015/0305370 A1
(43) Pub. Date: Oct. 29, 2015

(54) METHODS FOR MANAGING THE COMPOSITION OF DISTILLERS GRAIN CO-PRODUCTS

(71) Applicants: James Robert Bleyer, Maumee, OH (US); Jennifer L. Aurandt, Brighton, MI (US); Raymond Paul Roach, Midland, MI (US)

(72) Inventors: James Robert Bleyer, Maumee, OH (US); Jennifer L. Aurandt, Brighton, MI (US); Raymond Paul Roach, Midland, MI (US)

(21) Appl. No.: 14/697,745

(22) Filed: Apr. 28, 2015

Related U.S. Application Data
(60) Provisional application No. 61/985,267, filed on Apr. 28, 2014.

(51) Int. Cl. A23J 3/34; C12P 21/00; A23J 3/14

(52) U.S. Cl. CPC A23J 3/34; A23J 3/14; C12P 21/00

(57) ABSTRACT

The present invention provides for methods of managing the protein content of multiple distiller's grain co-products of a fermentation process. A valuable protein rich distiller's grain co-product having greater than 40 wt % protein on a dry basis can be removed from whole stillage or spent grains; however the protein content in the residual wet cake is reduced. The present invention provides for methods to mitigate protein depletion in wet cake by removing non-protein components from the wet cake stream, from the low protein thin stillage stream added to wet cake, or both streams. The present invention provides for blending protein depleted and protein enriched streams to meet the protein specifications of multiple distiller's grain co-products.

### 1. TECHNICAL FIELD

The present invention relates generally to processes for managing the protein content of multiple distiller's grain co-products produced in a fermentation process.

### 2. BACKGROUND ART

Fermentation processes produce many products, such as bio-chemicals, nutraceuticals, and bio-fuels. Xanthum gum is an example of a bio-chemical produced by the fermentation of carbohydrates by the bacteria Xanthomonas campestris. Many nutraceuticals are produced through fermentation processes utilizing bacteria, fungi, and algae. Ethanol is a biofuel produced through the fermentation of sugars into alcohol by the yeast Saccharomyces cerevisiae. The most common fermentation processes utilize sugars as the primary carbon source.

The sugars can be simple sugars from sugar producing plants such as sugar cane, sugar beets, and sweet sorghum. In the United States, most of the sugar used in fermentation is derived from grain starch. For example, ethanol is produced in a fermentation process by hydrolyzing starch to glucose and then converting the glucose to alcohol. Historically, corn has been the predominant grain used to produce ethanol, but other grains such as milo and wheat have also been used. The spent grain from the fermentation process is generally recovered as an animal feed. In the case of ethanol, the spent grain is generally referred to as distiller's grains.

In the case of corn ethanol, corn is ground and mixed with water to produce a slurry. The slurry is heated and treated with enzymes to convert the starch to monomer sugars. Yeast convert the sugars in the slurry to carbon dioxide (CO₂) and alcohol, resulting in an intermediate product known as beer. The CO₂ is vented or recovered as a by-product.

The alcohol is removed from the beer in a stripping column. The stripping column bottoms, referred to as “whole stillage” contain unfermentable components of the grain such as fiber, cereal proteins and lipids, yeast cells, unconverted starch and sugars, and secondary metabolites such as glycerol and organic acids.

Whole stillage is separated into a wet cake, also known as Wet Distiller's Grains (WDG), and thin stillage. A portion of the thin stillage is recycled to the front end of the plant as “backset” to reduce the need for fresh water. The remaining thin stillage is evaporated to produce a concentrate, sometimes referred to as distiller's solubles, or more commonly “syrup,” that can be sold and/or added to the wet cake to produce wet distiller's grains with solubles (WDGS). The wet cake with solubles can be sold as is but is typically dried to produce dried distiller's grain with solubles (DDGS). If syrup is not added to wet cake, the dried product is known as dry distiller's grains (DDG). Wet cake (WDG), WDGS, DDG, and DDGS are conventional distiller's grain products derived from stillage and are valuable animal feed products

Recently, there have been efforts made at producing an additional whole stillage co-product that has higher protein content than DDG or DDGS. Y. V. Wu, et al. (Cereal Chemistry 58(4) 343-347, 1981) and Y. V. Wu (Cereal Chemistry, 66(6), 506-509, 1989) describe the separation and isolation of a high protein fraction from corn ethanol whole stillage by a sequential process of filtration, centrifugal separation and dewatering and then drying, thereby achieving dry basis protein concentrations in the range of 42-57% depending on corn variety. U.S. Pat. No. 7,829,680, “System and Method for Isolation of Gluten as a Co-Product of Ethanol Production,” assigned to ProGold Plus Inc., discloses a process for separating a high protein fraction from whole stillage via screens and centrifuges. U.S. Patent Application Publication No. 2012/0121565, “Protein Recovery”, as applied for by AB Agri Ltd., discloses a process for “separating the majority of the suspended fibrous solids from the rest of the stillage; and then separating the majority of the protein containing fermentation agent from the water and dissolved solids.” U.S. Patent Application Publication No. 2014/0319066, “Thin Stillage Clarification,” as applied for by Yield and Capacity Group LLC, discloses a “low energy solids separation” process for clarifying stillage, especially thin stillage, which entails flocculating thin stillage suspended solids with a GRAS anionic polymer and recovering flocculated solids on a gravity fed belt filter. In separate downstream centrifugation steps, oil can be recovered from the flocculated solids and yeast can be recovered from the filtrate. U.S. Pat. No. 8,257,951, “Ethanol Production Process,” assigned to Little Sioux Processors, discloses a process for sequential micro-filtration and ultra-filtration of thin stillage to produce a protein/yeast rich co-product. U.S. Pat. No. 8,652,818, “Method for Extracting Protein from a Fermentation Product”, assigned to Poet Research, Inc., discloses a method for extracting zein protein from stillage. U.S. Patent Application Publication No. 2014/034259, “Protein Product,” as applied for by Valicor Inc., discloses a process for recovering a product having a protein content of 45.0% or more calculated by weight of dry matter by heating fermentation stillage to 200 degrees F.-350 degrees F. and separating a phase enriched in protein.

The present invention can also be applied to spent grains obtained prior to fermentation as are produced in alcoholic beverage fermentation processes and referred to commonly as brewers or distillers spent grains. Similar to the prior art for recovery of protein from whole stillage other prior art discloses recovery of protein rich co-products from spent grains. In U.S. Pat. No. 5,135,765 assigned to Kirin Beer K. K., Kishi et al. disclose a process for producing a protein-rich product and/or a fibrous product (wet cake) which includes the steps of milling brewer's spent grain (BSG) in a wet state, and sieving the milled BSG in the presence of water to thereby separate it into a protein-containing fraction (under-size sieved material) and a fibrous fraction (over-size sieved material).

In application WO2005/029974 A1 assigned to Heineken Technical Services B. V., K. Schwenke et al. disclose the separation of a proteinaceous juice from fermentation residue such as brewers or distillers spent grains by a process including sieving to separate fibers (wet cake) from protein-containing filtrate, followed by weight separation methods, e.g. settling and centrifugation to isolate a protein fraction from the sieve filtrate. The protein concentrate can be dried and compositions having 40-80 wt % protein on a dry basis are disclosed.

An important consideration when contemplating the removal from stillage or spent grains of a second co-product having high protein content is the impact on the protein content of the primary fiber-rich solid co-products such as wet cake, DDG, or DDGS. In conventional dry-grind ethanol plant operations, syrup is added back to wet cake and the mixture is dried to produce DDGS. Prior to syrup addition, wet cake crude protein is as high as 36 wt % on a dry matter basis [Kim et al. Bioresource Technology 99 (2008) 5165-5176]. Although syrup contains yeast and cereal proteins, the large amount of non-protein components in syrup including minerals, glycerol and organic acids lead to DDGS having a diluted protein content of 26-30 wt %. Consequently processes designed to remove additional protein from whole stillage can exacerbate the challenges of managing protein in wet cake derived products. Therefore there is a need for methods to produce a high protein product (>40 wt % crude protein on a dry matter basis) from stillage and simultaneously manage the protein content of wet cake derived products such as WDG, DDG and DDGS.

In an exemplary embodiment of the present invention, protein is removed from thin stillage to produce a high-protein co-product; subsequently, non-protein components are removed from the reduced protein thin stillage, a syrup is produced by evaporation thereof, and the syrup is added back to wet cake. Whereas adding reduced protein syrup to wet cake would exacerbate protein dilution in DDGS, this problem is mitigated by removing non-protein components from the reduced protein thin stillage. The prior art discloses some methods of removing non-protein components from thin stillage including anaerobic digestion, precipitation of minerals or inorganic compounds, micro-filtration membranes and combinations thereof. Of these, anaerobic digestion is presently used in commercial ethanol plants to treat thin stillage evaporator condensate by removing small percentages of soluble volatile organic compounds such as short chain alcohols, acids and aldehydes.

In U.S. Pat. No. 8,153,006, assigned to Procorp Enterprises LLC, Fessler et al. disclose a process for treating thin stillage from an ethanol production process by an anaerobic digester system equipped with an external solids/liquid separator such as an ultrafiltration (UF) membrane unit. Ammonia rich liquid permeate can be obtained from the UF unit and optionally recycled to the digester, recycled to the ethanol fermentation process in lieu of fresh water and ammonia or used to produce a fertilizer such as magnesium-ammonium-phosphate (“struvite”). Although Fessler et al. disclose treatment of thin stillage by anaerobic digestion, and return of ultra-filtered digester effluent to the ethanol process as a process water stream, they do not disclose production of a high-protein distiller's grain co-product or the use of digester effluent as a means to manage protein content in multiple distiller's grain co-products.

In U.S. Pat. No. 8,669,083, assigned to Eisenmann Corp., Veit et al. disclose a process for the anaerobic digestion of thin stillage (and optionally syrup), thereby producing biogas and a liquid effluent stream. Effluent from anaerobic digestion can be recycled as backset to the pre-treatment (i.e. liquefaction/saccharification) section of the fermentation plant and reduces the usual amount of thin stillage backset. Veit et al. do not disclose the addition of effluent to distiller's grains or production of a high protein co-product from thin stillage, nor the use of anaerobic digestion as a means of managing the protein content of multiple distiller's grain co-products.

In European Patent Application EP 2581439 A1 as applied for by Agraferm Technologies AG, H. Freidman discloses a process for treatment of ethanol stillage comprising the steps of separating stillage by for example a decanting centrifuge, membrane filter unit, screw press, drum filter and/or drum screen, into a thin fraction and a thick fraction and separately digesting the fractions. Freidman discloses that the thin fraction can be digested much more quickly than the thick fraction and hence the thin fraction digester can be of much smaller volume. The thin fraction need not be devoid of suspended solids as the upflow digester specified by Freidman is designed without pore-containing materials or filters. Freidman discloses a downstream “nitrogen sink” system to remove ammonia as a gas from the digestate and use of said ammonia to enrich solid and liquid fertilizer co-products. Freidman further discloses that the purified water resulting from digestion can be returned to the “ethanol plant.” Freidman does not disclose the separation of a high-protein co-product from the thin fraction or the use of anaerobic digestion as a means to manage the protein content of multiple distiller's grain co-products. Freidman does not disclose addition of digestate or effluent to wet cake or production of an animal feed thereof.

In European Patent Application EP 1790732A1 as applied for by Prokop Invest AS and others, Prochozka et al. disclose the comprehensive use of ethanol production stillage to give multiple end products including dried stillage with low salt content, granulated sludge from anaerobic digestion, solid fertilizer as struvite, elementary sulfur and waste heat. Prochazka et al. disclose a two stage separation of solids from raw stillage. In the first stage cake is separated from “raw” stillage by decantation centrifugation. Residual particles, especially cereal proteins, are removed from the decanter centrate by a method such as air flotation, centrifugation, vacuum filtration or combinations thereof. Prochazka et al. disclose that the removal of residual solids protects the anaerobic biomass granules from disintegration and that the protein sludge removed in this step can be dewatered and combined with the first stage cake to increase the nitrogenous content of the final dry animal feed produced thereof. Liquid fractions from both stillage separation steps are blended and acidified under controlled conditions at pH ranging between 4.8 and 9.2. The resulting mixture is then treated anaerobically with granulated acetogenic and methanogenic bacteria. The accumulated granulated sludge is removed and stored for sale. The biogas is treated to remove sulfur and then used for energy production. From the digester liquid fraction, nitrogenous substances are removed by dosing magnesium chloride and phosphoric acid resulting in precipitation of struvite that is separated and removed as a high-quality fertilizer. The liquid fraction is subsequently taken to aerobic final treatment where sludge is separated. After having been thickened, the sludge can be used in agriculture. Prochazka et al. disclose production from stillage of a second distiller's grain product having high protein content and addition of this stream to conventional wet cake to increase the nitrogen content of animal feed made thereof. Prochazka et al. do not disclose addition of digester effluent to wet cake as a means to improve feed nitrogen content, but rather that the nitrogen can be removed as magnesium ammonium phosphate (struvite). The remaining liquid effluent is treated aerobically to produce purified water for discharge or re-use in the fermentation process.

In U.S. Patent Application No. 2014/0065685, G. Rosenberger et al. disclose a process for the treatment of thin stillage from an ethanol fermentation process using an anaerobic membrane bioreactor. The membrane bioreactor produces a highly clarified permeate that can be recycled as backset to the fermentation process without contributing suspended solids which would otherwise necessitate a reduction in the fresh feedstock solids charged to the fermenter. Rosenberger et al. do not disclose addition of digestate to wet cake or production of an animal feed thereof, or recovery of a high-protein distiller's grain co-product from the fermentation stillage nor the management of protein levels in multiple distiller's grain co-products.

In U.S. Patent Application No. 2014/0134697A1 as applied for by DSM IP Assets B. V., H. L. Bihl et al. disclose the digestion of organic materials, including fermentation waste such as brewers spent grains, to biogas. The process is a two-stage process whereby in the first stage the organic material is heat treated to pasteurize and then enzymatically treated with proteases and/or lipases and/or cellulases which respectively digest proteins, lipids and complex carbohydrates. The effluent of the first stage is separated into a liquid and a washed solid fraction. The liquid fraction is fed to the second stage, an anaerobic digestion process to produce biogas. Although Bihl et al. disclose treatment of a fermentation waste stream in a manner which affects protein content, the production of a high-protein distiller's grain co-product and management of protein content in multiple distiller's grain co-products are not disclosed.

In U.S. Pat. No. 8,017,365 Rein et al., disclose a “process resource production system” to convert an ethanol byproduct such as whole stillage, thin stillage and thin stillage solubles (i.e. thin stillage with suspended solids removed) to coproducts including an inorganic fertilizer such as struvite, and three products from anaerobic digestion: biogas, biosolids (an organic fertilizer) and a liquid stream suitable for treatment to produce recycle water. Rein et al. disclose an embodied two-step process in which high protein solids are first removed from thin stillage and then oil is removed by adjusting pH to approximately 6 and separating the oil by a density separator. The high protein solids removed from thin stillage can be combined with DWG to enhance protein content. Rein et al. also disclose that the anaerobic digester effluent (including biosolids) can be sent to the ethanol plant evaporators for thickening and that the thickened biosolids can be added to DWG. Rein et al. do not disclose producing a separate dry protein co-product from thin stillage nor the management of protein content in multiple distiller's grain co-products.

In U.S. Patent Application Publication No. 2010/0196979 as applied for by BBI International Inc., Birkmire et al. disclose a process for converting brewers spent grains and other brewery biomass streams into cellulosic ethanol and other products such as pelletized fuel, biogas (via anaerobic digestion) and livestock feed. Birkmire et al. disclose conversion of brewery biomass streams including spent grains by a process of cellulosic pretreatment, enzymatic hydrolysis, fermentation to ethanol and ethanol separation by distillation and dehydration. Residual solid slurry from fermentation is separated by centrifugation into wet cake and the liquid centrate. The centrate can be clarified to concentrated syrup (retentate) and clean water stream (permeate) via membranes, or anaerobically digested to produce biogas. Retentate syrup can be added to the wet cake and dried to produce an animal feed. It is disclosed that the purified water resulting from digestion can be returned to the “ethanol plant.” Birkmire et al. do not disclose recovery of a high-protein distiller's grain co-product from the fermentation stillage nor the management of protein levels in multiple distiller's grain products. Birkmire et al. do not disclose addition of digestate to wet cake or production of an animal feed thereof.

Others have disclosed systems that utilize membranes or combinations of membranes and anaerobic digestion. In U.S. Pat. No. 7,267,774 assigned to NouVeau Inc. (USA), Peyton et al. disclose a potable water or beverage product obtained by treating still bottoms in an ethanol production facility by means of membrane pressure filtration (ultrafiltration, nanofiltration, reverse osmosis) and anaerobic digestion. An objective of Peyton et al. is to capture the mineral and nutrient content of the fermentation process in the water or beverage product. Whole stillage can be separated by decanting centrifugation to remove large solids prior to UF-NF-RO filtration. Anaerobic digestion of combined solids from stillage and the concentrate streams from UF-NF-RO filtration produces a biogas with sufficient energy to power the pressurized filtration system. Also key to Peyton et al. is maintaining the stillage warm so as to preserve its pasteurized state. Peyton et al. disclose that a portion of the pressure filtration permeate can be added to distiller's grains as a means of controlling solids concentration in the digester feed stream; however, they do not disclose production of a high protein co-product from thin stillage nor the management of distiller's grains protein content via their process.

In U.S. Pat. No. 5,250,182 assigned to Zenon Environmental Inc. (Canada), Bento et al. disclose a process for removal of glycerol and lactic acid from thin stillage by means of UF, NF and RO membrane units in series. The UF and NF units retentate streams (referred to as “concentrates” in Bento et al.) are concentrated in fine insolubles and soluble proteins having MW>20,000 Daltons. Glycerol, lactic acid and dissolved minerals permeate the UF freely, are minimally rejected by the NF unit, substantially rejected by the RO unit, and are thus collected in the RO retentate stream. The UF and NF concentrates are thus enriched in proteins by virtue of lactic acid, glycerol and minerals removal. Bento et al. disclose that the UF and NF concentrates can be added to centrifuged still bottoms solids (i.e. wet cake recovered from whole stillage) to produce an animal feed that contains essentially all of the proteins (>1000 Daltons) that were present in whole stillage. Bento et al. further disclose that without mixing with solids from the centrifuged still bottoms, the UF and NF concentrates can be used to prepare a proteinaceous feed for fish. Although Bento et al. disclose, the production of a second high-protein distiller's grain co-product via their membrane process, they do not disclose a method of maintaining protein content in the wet cake co-product.

W. H. Kampen in U.S. Pat. No. 5,177,008 discloses a process for clarification of thin stillage by micro-filtration and recovery of fermentation by-products such as glycerol, organic acids, betaine, potassium sulfate and distiller's dry grains having improved flowability owing to removal of sticky glycerol. Kampen discloses that stillage can be separated by centrifugation and micro-filtration and optionally treats the permeate with proteases prior to further separations. The micro-filtration retentate containing larger particles goes to fertilizer or animal feed processing. The permeate can be further processed by chromatographic separation and physical separations (e.g. distillation and crystallization) to recover glycerol, organic acids, potassium sulfate and betaine. Although Kampen discloses removal of various non-protein components from stillage via membrane separation and protease treatment of permeate, no such treatments and processes as means of managing protein levels in multiple distiller's grain co-products are disclosed.

Non-protein components can also be removed by conversion to biomass in aerobic processes. Examples of growth of fungal biomass ranging from yeast to filamentous fungi and even macrofungal species have been reported. J. van Leeuwen et al. in U.S. Pat. No. 8,481,295 disclose the growth of filamentous fungi on alcohol fermentation stillage to produce a high-value fungal biomass that can be recovered and used as an animal feed, human food or as a source of nutraceuticals. The fungal processing removes organic substances from the water that are otherwise inhibitory to the reuse prospects for the water, i.e. suspended and dissolved organic matter, including glycerol, lactic and acetic acids. In a review of the treatment of distillery and brewery waste by yeasts, filamentous fungi, white-rot fungi and mixed cultures the concomitant benefits of effluent purification and production of fungal biomass rich in protein are disclosed (H. Singh, Remediation of Distillery and Brewery Wastes, Chapter 3 in Mycoremediation—Fungal Bioremediation, Wiley Publishing, 2006).

A second means of managing protein content of distiller's grain coproducts is to remove non-protein components from wet cake. One will appreciate that this approach can be applied as an alternative or supplemental to removing non-protein components from de-proteinated thin stillage. Cellulose and hemicellulose fibers are non-protein components that can be removed from wet cake to manage the protein content of distiller's grain co-products. The methods of chemical and/or enzymatic hydrolysis of cellulose and hemicellulose are well known to those skilled in the art and comprise a key step in the overall process of converting such fibers to cellulosic ethanol. The prior art commonly refers to processing of “lignocellulosic biomass” to ethanol. It is noted that the lignin content of a particular biomass feedstock can be high, as in the case of hardwoods, or low as in the case of corn or corn ethanol stillage. More generally, the prior art simply refers to such feedstocks as “cellulosic,” regardless of the specific lignin, cellulose and hemicellulose composition and “cellulosic ethanol” is derived thereof. Regardless of feedstock nomenclature, it is the cellulose and hemicellulose fractions that serve as sugar sources for downstream recovery and conversion to alcohol. The overall cellulosic ethanol process consists of four major unit operations: pretreatment, hydrolysis, fermentation, and product separation/purification. Pretreatment is required to alter the biomass macroscopic and microscopic size and structure as well as its submicroscopic chemical composition and structure so that hydrolysis of the carbohydrate fraction to monomeric sugars can be achieved more rapidly and with greater yields. Hydrolysis includes the processing steps that convert the carbohydrate polymers into fermentable monomeric sugars (Wyman, 1999). Cellulose is hydrolytically broken down into glucose either enzymatically by cellulases or chemically by sulfuric or other acids. Hemicellulases or acids hydrolyze the hemicellulose polymer to release its component sugars. Glucose, galactose, and mannose, six carbon sugars (hexoses), are readily fermented to ethanol by many naturally occurring organisms, but the pentoses xylose and arabinose (containing only five carbon atoms), are fermented to ethanol by only a few native strains. Hence, by appropriate selection of pretreatment, organism type and process configuration, one can choose to produce and convert sugars produced from cellulose and/or hemicellulose. In some cases, no pretreatment is required. For example, Kim et al. (Bioresource Technology 99 (2008) 5206-5215) showed that 60-70% of the available glucan in non-pretreated DDGS can be digested with cellulase only. As expected, cellulose digestion of glucan improves to greater than 90% when DDGS are pretreated by liquid hot water (LHW) or ammonia fiber expansion (AFEX).

Genetically modified organisms are being developed which can simultaneously hydrolyze biomass and convert the sugars to ethanol. Chung et al. reported engineering Caldicellulosiruptor bescii for ethanol production (PNAS 111 (24), 8931-8936). C. bescii is an anaerobic thermophilic cellulolytic bacterium which grows optimally at −80° C. and has the ability to use a wide range of substrates, such as cellulose, hemicellulose, and lignocellulosic plant biomass without harsh and expensive chemical pretreatment. Chung et al. demonstrated direct conversion of unpretreated switchgrass to ethanol with an engineered C. bescii strain.

P. E. V. Williams in international Patent Publication No. WO2013/021161 A2, as applied for by AB Agri Ltd, discloses a process for recovering two high protein co-products from a fermentation byproduct (e.g. whole stillage) comprising separating whole stillage into a wet cake fraction having a high content of fiber and a thin stillage fraction high having a high content of fermentation agent (yeast). Protein (substantially yeast associated protein) is separated from the thin stillage by for example disk stack centrifugation. A portion of the fiber in the wet cake may be solubilized by for example chemical or enzymatic hydrolysis and the solubilized fiber separated from residual non-fermentable high protein solids. Williams discloses that due to previous saccharification, fermentation and distillation process, the cell wall polysaccharides are more amenable to enzymatic digestion and/or chemical modification. Williams further discloses that the high protein solids derived from solubilization of the wet cake fiber contain substantially more protein than conventional DDGS, e.g. greater than 40% crude protein on a dry matter basis. The solubilized fiber can be recycled to fermentation or an anaerobic digestion step. Williams discloses that wet cake fiber solubilization can be performed without any additional intervening treatment steps.

In U.S. Pat. Nos. 8,759,050 and 8,633,003 assigned to Quad County Corn Processors, T. Brotherson discloses a method of producing ethanol from whole stillage, generally comprising thermally and chemically pretreating the whole stillage to hydrolyze hemicellulose, for example by acidification with heating under pressure, followed by enzymatic treatment to hydrolyze cellulose and subsequent fermentation of the hydrolysis sugars to ethanol. Brotherson further discloses that the required severity of the pretreatment (pH, time, temperature) is generally first determined by the desire for hemicellulose hydrolysis. If little to no hemicellulose hydrolysis is required, the severity of the pretreatment is determined by the desired yield of glucose from cellulose during the enzymatic step (col 4, lines 58-60). Brotherson elaborates that although hemicellulose hydrolysis is not necessary, it however provides advantages via improved feed product drying, reduced stillage viscosity (less pumping energy) and improved release of oil from stillage. Brotherson further discloses that after fermentation of the cellulose hydrolysate sugars, the resulting stillage produces DDGS having a higher protein content than conventional dry grind ethanol DDGS. Brotherson does not disclose that a second high protein co-product can be derived from thin stillage or the management of protein content of distiller's grain co-products.

In summary, removing protein from stillage or spent grains results in a reduced protein wet cake, and thus DDGS with reduced protein levels and reduced value. In the prior art, the deleterious effects of the removal of the protein from stillage on the protein content of wet cake and DDG or DDGS has either gone unrecognized or unmitigated. There remains a need for processes that produce high value protein co-products from stillage while mitigating protein depletion in DDG and DDGS.

### SUMMARY OF THE INVENTION

The present invention provides for a method of managing the protein content of multiple distiller's grain co-products of a fermentation process by separating whole stillage or spent grains into wet cake and thin stillage, removing a protein rich distiller's grain co-product having greater than 40 wt % protein on a dry matter basis from the thin stillage and producing a reduced protein thin stillage, removing at least some non-protein components from the reduced protein thin stillage, and adding all or a portion of the reduced protein thin stillage, having at least some non-protein components removed, to at least some of the wet cake.

The present invention also provides for a method of managing the protein content of multiple distiller's grain co-products of a fermentation process by separating whole stillage or spent grains into wet cake and thin stillage, removing a protein rich distiller's grain co-product having greater than 40 wt % protein on a dry matter basis from the thin stillage and producing a reduced protein thin stillage, and removing at least some non-protein components from the wet cake and producing a protein enriched wet cake.

The present invention provides for methods of managing the protein content of multiple distiller's grain co-products of a fermentation process by separating whole stillage or spent grains into wet cake and thin stillage, separating at least some of the thin stillage into a high protein stream and a low protein stream, producing a high protein distiller's grain co-product from the high protein stream, removing and recovering additional protein components from the low protein stream and adding the additional recovered protein components to the wet cake.

The present invention provides for a method of managing protein by separating whole stillage or spent grains into wet cake and thin stillage, removing a protein rich distiller's grain co-product having greater than 40 wt % protein on a dry matter basis from the thin stillage and producing a reduced protein thin stillage, adding a micro-organism to the reduced protein thin stillage and growing biomass having higher protein content on a dry weight basis than wet cake, consuming non-protein components of the reduced protein thin stillage during the course of growing the biomass, and harvesting the biomass.

The present invention further provides for a system of managing the protein content of multiple distiller's grain co-products in a grain fermentation facility by separating whole stillage or spent grains into wet cake and thin stillage, removing a protein rich distiller's grain co-product having greater than 40 wt % protein on a dry matter basis from the thin stillage and producing a reduced protein thin stillage, removing non-protein components from a product chosen from the group consisting of the reduced protein thin stillage, the wet cake, and both the reduced protein thin stillage and the wet cake, and achieving a desired protein content in multiple distiller's grain co-products by combining protein containing streams chosen from the group consisting of thin stillage, wet cake, reduced protein thin stillage, reduced protein thin stillage having non-protein components removed, wet cake having non-protein components removed, protein rich distiller's grain co-product, biomass grown on reduced protein thin stillage, evaporated concentrates of substantially liquid streams in the preceding list, and dried forms of substantially wet solids in the preceding list.

### BRIEF DESCRIPTION OF THE DRAWINGS

Other advantages of the present invention are readily appreciated as the same becomes better understood by reference to the following detailed description when considered in connection with the accompanying drawings wherein:

FIG. 1 is a flowchart of prior art grain based fermentation;

FIG. 2 is a flowchart of prior art grain based fermentation with removal of high protein co-product from stillage;

FIG. 3 is a flowchart of the present invention with removal of a high protein co-product from stillage, removal of non-protein components from the low protein stream by anaerobic digestion and adding the treated low protein stream to wet cake;

FIG. 4 is a flowchart of the present invention with removal of a high protein co-product from stillage, removal of non-protein components from the low protein stream by aerobic digestion and growth of biomass;

FIG. 5 is flowchart of the present invention wherein the protein content of the wet cake is increased by adding one or more chemicals to precipitate low protein components from the low protein stream;

FIG. 6 is a flowchart of the present invention with removal of a high protein co-product from stillage wherein additional protein is removed from the low protein stream and the additional protein is added to the wet cake; and

FIG. 7 is a flowchart of the present invention with removal of a high protein co-product from stillage wherein the protein content of the wet cake is increased by hydrolyzing and removing fiber hydrolysate from the wet cake; and

FIG. 8 is a flowchart of the present invention with removal of a high protein co-product from stillage wherein non-protein components are removed from the low protein stream and wet cake.

### DETAILED DESCRIPTION OF THE INVENTION

The following detailed description of embodiments of the invention references the accompanying drawings. The embodiments are intended to describe aspects of the invention in sufficient detail to enable those skilled in the art to practice the invention. Other embodiments can be utilized and changes can be made without departing from the scope of the claims. The following detailed description is, therefore, not to be taken in a limiting sense. The scope of the present invention is defined only by the appended claims, along with the full scope of equivalents to which such claims are entitled.

In this description, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated or as will be readily apparent to those skilled in the art from the description. For example, a feature, structure, act, etc. described in one embodiment can also be included in other embodiments, but is not necessarily included. Thus, the present technology can include a variety of combinations and/or integrations of the embodiments described herein.

Certain terms used throughout this description are taken to have the meanings defined below.

“Spent grains” as used herein refers to a product stream of an alcoholic beverage fermentation process in which the starch portion of grain has been converted to soluble fermentable sugars and carbohydrates by a cooking and/or enzymatic process, said fermentable sugars and carbohydrates separated as a liquid from the starch-depleted grain and transferred to the fermentation step. Spent grains from beverage fermentation processes are commonly referred to as brewer's or distiller's spent grains. Spent grains can be suspended in water to produce a slurry similar to whole stillage.

“Stillage” as used herein, refers to a cloudy liquid produced during fermentation that includes solids that are not fermentable, solubles, oils, organic acids, salts, proteins, and various other components.

“Whole stillage” as used herein, refers to a resultant product stream in which the primary products of grain fermentation, e.g. an alcohol, have been stripped from the stream and before the stream is acted upon by any other process.

“Thin stillage” as used herein, refers to a resultant product stream in which some or all of the insoluble solids have been removed from whole stillage. Insoluble solids can be removed from the whole stillage by centrifugation, filtration, settling or any other suitable mechanism.

“Wet cake” or “wet distiller's grains” (WDG) as used herein, refers to insoluble solids removed from whole stillage including the liquid and soluble solids that remain with the insoluble solids after separation.

“Distiller's grain co-products” as used herein refers to the category of fibrous or proteinaceous products which may be derived from fermentation spent grains. In the dry-grind fuel ethanol process, examples include wet cake (DWG), protein enriched DWG, DDG, DDGS. For clarity in the present specification, distiller's corn oil is not included in this category. In other fermentation process such as beverage alcohol, grain is also used as a source of sugar. Once the starch in the grain has been converted to sugar, the spent grain is typically removed from the process before fermentation. Spent grains from these operations are referred to by various names including “brewer's spent grain” (BSG) or “distiller's spent grain” (DSG). When used herein, the terms distiller's grain co-products and dried distiller's grains includes spent grains from any fermentation process, including those where the spent grains are removed prior to fermentation.

“High protein solids” as used herein, refers to a stillage fraction that contains a higher level of protein on a dry weight basis than whole stillage or spent grains.

“Fermentation” as used herein, refers to a biological process, either anaerobic or aerobic, in which suspended or immobilized micro-organisms or cultured cells in a suitable media are used to produce metabolites and/or new biomass.

“Off product metabolites” as used herein, refers to metabolites produced during a fermentation process other than those products targeted for production by the fermentation process.

“Protein” as used herein, refers to organic molecules, which can be soluble or insoluble, including individual amino acids, short and long peptide chains, or proteins.

“Protein depleted stream” as used herein, refers to a stream in which some or all of the protein has been removed.

“Low protein stream” as used herein, refers to a stream that has a lower protein content, including no protein, than the stream from which it was extracted.

“High protein stream” as used herein, refers to a stream that has a higher protein content than the stream from which it was extracted.

“Non-protein component(s)” as used herein, refers to soluble or in-soluble solids including fiber, simple and complex carbohydrates, lipids, phospholipids, nucleic acids, organic acids, alcohols, inorganic minerals and salts and excludes those components described as “protein” above.

Most generally, the present invention provides for a method of producing a high protein distiller's grain co-product, containing greater than 40 wt % crude protein on a dry matter basis, from stillage or spent grains and simultaneously managing the protein content of distiller's grain products derived from wet cake such as WDG, DDG and DDGS. The present methods provide for separating whole stillage or spent grains into wet cake and thin stillage, and separating thin stillage into a high protein distiller's grain co-product and reduced protein thin stillage. Subsequently, at least some of the reduced protein thin stillage or at least some of the wet cake or both can be treated separately to remove non-protein components. Treated reduced protein thin stillage can be concentrated to syrup. Plant owners can produce and combine standard thin stillage syrup, reduced protein thin stillage syrup, treated reduced protein thin stillage syrup, standard wet cake, and treated wet cake in any proportions to achieve a desired protein content in the distiller's grain products derived from wet cake such as WDG, DDG, and DDGS.

More specifically, a method of managing the protein content of multiple distiller's grain co-products of a fermentation process is provided by separating whole stillage or spent grains into wet cake and thin stillage, removing a protein rich distiller's grain co-product having greater than 40 wt % protein on a dry matter basis from the thin stillage and producing a reduced protein thin stillage, removing at least some non-protein components from the reduced protein thin stillage, and adding all or a portion of the reduced protein thin stillage, having at least some non-protein components removed, to at least some of the wet cake.

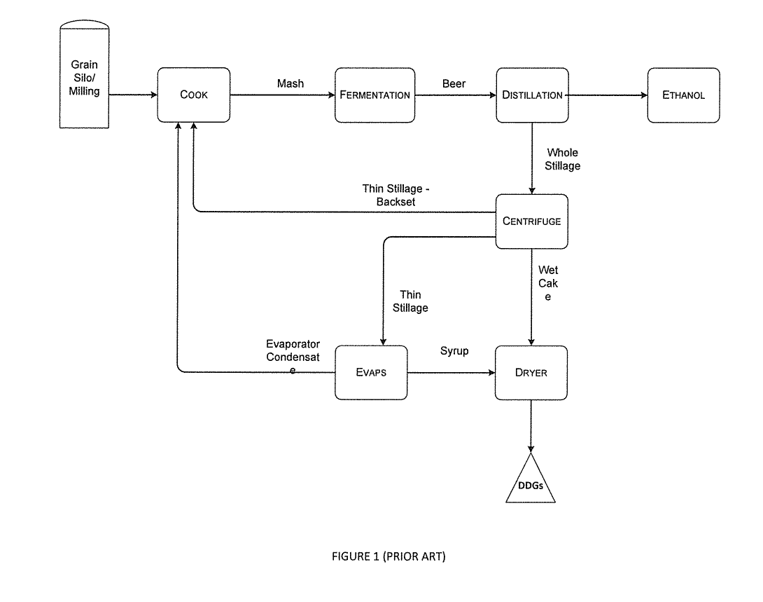

As representative of a grain fermentation process, a dry-grind ethanol process is depicted in FIG. 1 (labeled Prior Art). In the dry-grind process, whole kernel corn is milled to flour and slurried. The slurry is treated with one or more enzymes to convert the starch in the slurry to sugars creating a fermentation mash. An organism such as yeast is added to the mash to convert the sugars to ethanol. The ethanol is stripped from the slurry to produce whole stillage. Whole stillage is recovered and separated into wet cake and thin stillage. In U.S. dry-grind ethanol plants, the decanting centrifuge is the most common whole stillage separation device although any suitable solid-liquid separation mechanism, including, but not limited to centrifuge, filtering centrifuge, vibrating screen, pressure screen, paddle screen, filter, and membrane or combinations thereof can be applied. A portion of the thin stillage known as backset is recycled to the front end of the plant as make-up water for slurrying fresh corn. The balance of the thin stillage is evaporated to syrup in a multi-effect evaporator. Corn oil is commonly recovered from the concentrated thin stillage by centrifugation at an intermediate stage of evaporation. Various chemicals such as demulsifiers can be added to enhance oil separation. Evaporator condensate is also a source of front end recycle water; however, many plants first treat condensate by anaerobic digestion to remove various volatile organic compounds deemed to be inhibitory to fermentation. Syrup from the last stage of evaporation can be sold as is but more commonly it is added to wet cake and sold either wet as wet distiller's grains with solubles (WDGS), or most commonly, dried to produce DDGS having less than 15% moisture.

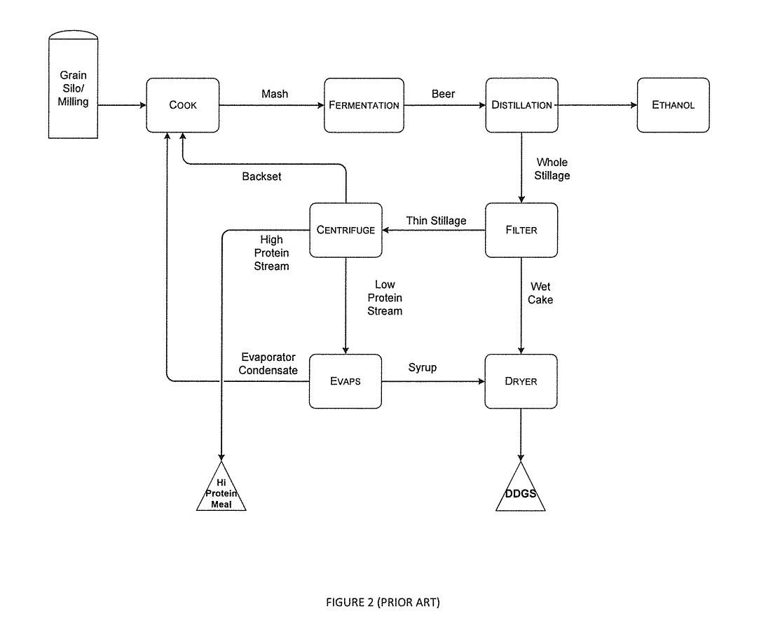

Although not widely implemented across the corn ethanol industry, processes have been developed to further separate thin stillage into a high protein stream (protein rich distiller's grain co-product) and a low protein stream (reduced protein thin stillage), as previously referenced. FIG. 2 (labeled Prior Art) depicts a simple process for separation of thin stillage into high and low protein containing streams. The thin stillage can be separated by any suitable mechanism, including, but not limited to adding one or more protein agglomerating chemicals, decanting centrifuge, disc stack centrifuge, nozzle disc centrifuge, filtration, filtering centrifuge, pressure screen, paddle screen, gravity belt filter, membrane filters, dissolved air flotation, and combinations thereof. The recoverable volume of the high protein stream and its relative protein content are influenced both by the whole stillage separation process and the subsequent thin stillage separation process. For example, as disclosed by ProGold in U.S. Pat. No. 7,829,680, a system of pressure filters and counter-current washing provides enhanced transfer of protein from whole stillage into the thin stillage yielding protein enriched thin stillage. Subsequently, ProGold discloses for example, that a centrifuge or centrifuge assembly can be used to separate the thin stillage into a protein fraction, an oil fraction and a fraction rich in water phase and minerals.

The high protein co-product stream can be dried to produce a high protein meal. The wet or dry high protein co-product stream is suitable for animal feed and as an organic fertilizer.

The low protein stream can be concentrated by for example evaporation as is done for conventional thin stillage; however this would result in syrup having lower protein content than conventional thin stillage syrup. Addition of a low protein syrup to wet cake or possibly reduced protein wet cake will reduce the protein content and hence the value of WDGS or DDGS produced thereof. According to the present invention, the deleterious effect of adding a low protein stream to wet cake is mitigated by either removing non-protein components from the low protein stream, removing non-protein components from the wet cake, or a combination thereof.

The low protein stream contains soluble and insoluble solids. The soluble solids are primarily minerals and soluble solids from the fermentation agent or feedstock including organic compounds, soluble proteins, residual sugars, and off-product metabolites. For example, in corn ethanol fermentation, yeast will produce off-product metabolites such as glycerol and organic acids. Any of these non-protein components can be removed from the low protein stream. The removed non-protein components, if not destroyed in the removal process, can be optionally recovered for other uses.

Biological Removal of Non-Protein Components from Low-Protein Stream

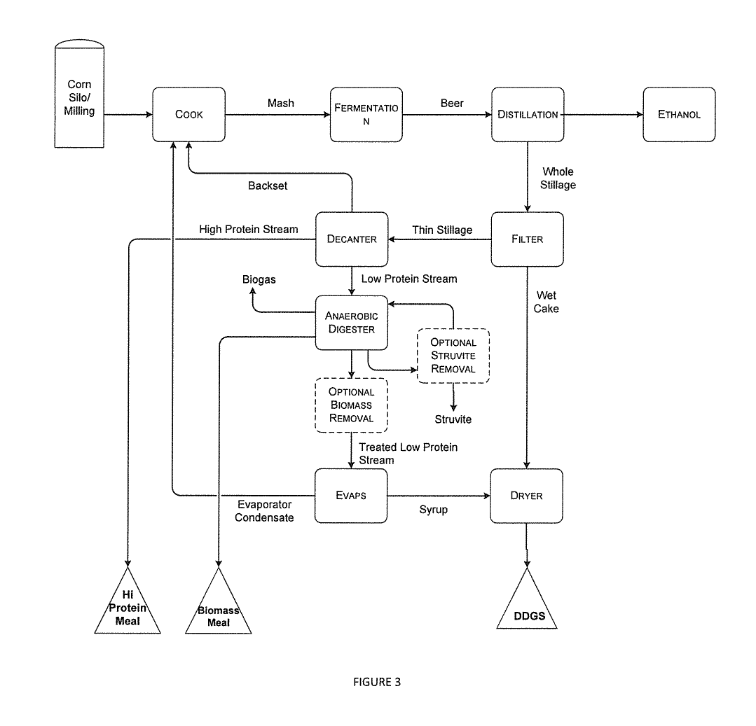

In one embodiment of the present invention, removal of the non-protein components from reduced protein thin stillage can be accomplished by anaerobic digestion as shown in FIG. 3. In the case of anaerobic digestion, one or more anaerobic organisms consisting of acetogens and methanogens are added to the low protein stream to preferentially metabolize the non-protein components, especially glycerol, residual simple carbohydrates and organic acids. The biogas product of anaerobic digestion consists primarily of methane and carbon dioxide and after gas cleaning can be a useful energy source. Following anaerobic digestion of the low protein stream, the digester effluent, with or without organisms, is then collected, concentrated and added to the wet cake to produce WDGS which can be further dried to form DDGS. The present invention provides for the WDGS and the DDGS produced herein.

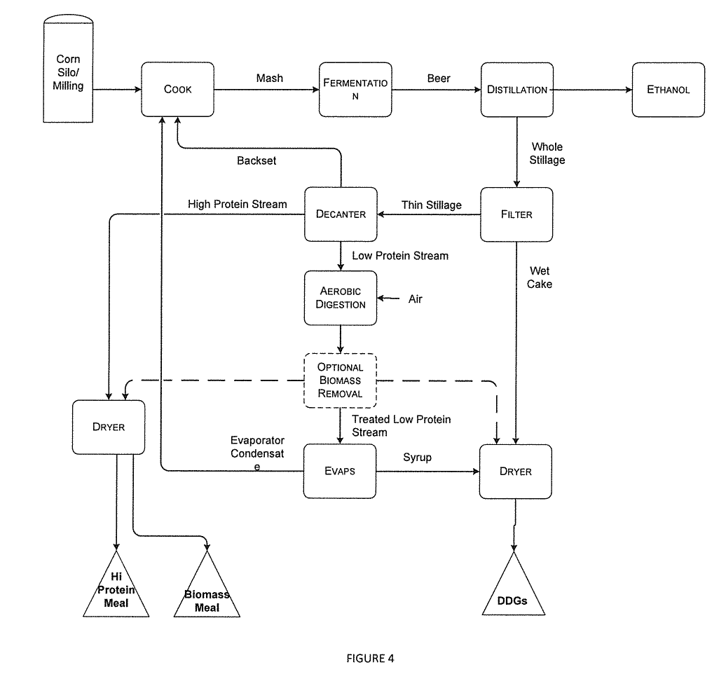

In another embodiment, as shown in FIG. 4, aerobic fermentation/digestion can be used to remove non-protein components. Whole stillage is collected from a fermentation process and separated into wet cake and thin stillage. The thin stillage is further separated into a high protein stream and a low protein stream. The low protein stream is collected and organisms are added, oxygen is provided to maintain a desired dissolved oxygen level, and the organisms preferentially metabolize the non-protein components in the stream. The low protein stream can be concentrated prior to the addition of the organisms. Growth of organisms during aerobic digestion creates additional biomass with a protein level higher than the low protein stream. The organisms can also produce protein as a metabolite. The biomass can be recovered separately as a high protein product, added to the high protein stream or added to wet cake and recovered as WDGS or DDGS. The resultant biologically treated low protein stream is added to the wet cake. The resulting biological treated low protein stream can be concentrated before adding to the wet cake. The wet cake can be dried before or after the addition of the biologically treated low protein stream. The organisms can be removed from the biologically treated low protein stream before adding to the wet cake.

The low protein stream can be used as a medium in a secondary fermentation. The protein level in the low protein stream is raised by either metabolizing non-protein components or selecting organisms that are high in protein or produce metabolites that are high in protein. The secondary fermentation can be aerobic or anaerobic and can produce biomass or metabolites. The organisms used for the secondary fermentation include, but are not limited to: bacteria, algae and fungus, including yeast.

In other embodiments of removing non-protein components from the low protein stream, the low protein stream is concentrated prior to biological digestion. The effluent of the low protein stream digestion can also be concentrated prior to adding to at least some of the wet cake. The methods can further include drying at least some of the wet cake before or after addition of the low protein stream, using the at least some of the modified wet cake as a component of an animal feed, a component of human food, and combinations thereof.

Non-Biological Removal of Non-Protein Components from the Low-Protein Stream

Removal of at least some of the non-protein components from the low-protein stream can also be accomplished non-biologically by adding one or more chemicals, mineral precipitation, filtration, microfiltration membranes, ultrafiltration membranes, nanofiltration membranes, reverse osmosis membranes, ion exchange, dissolved air floatation, quiescent decantation, decanting centrifuge, disc stack centrifuge, nozzle disc centrifuge, filtering centrifuge, paddle screen, and combinations of any of these removal mechanisms. This removing step (either biological or non-biological removal) can be performed either before concentrating the reduced protein thin stillage or after concentrating the reduced protein thin stillage. Concentrating can be performed with a multi-effect evaporator.

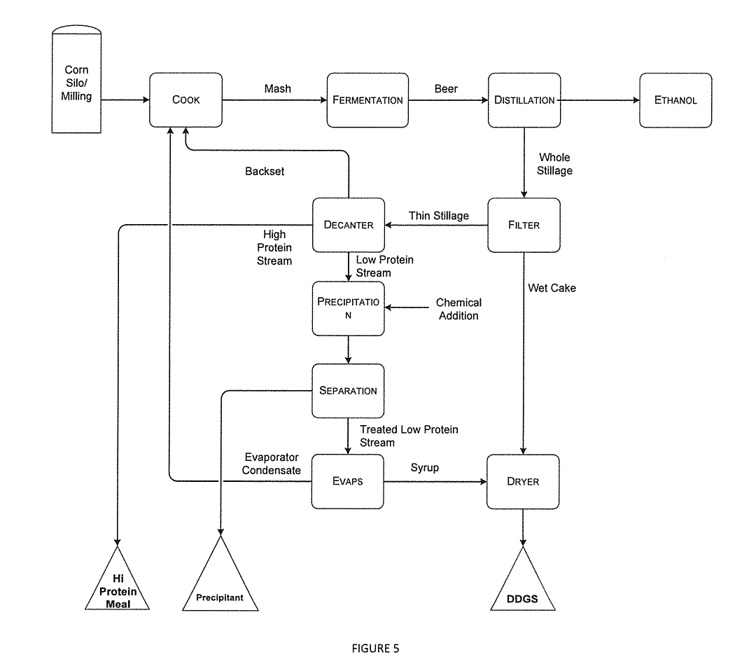

In an embodiment of the present invention, the low protein components are removed by precipitation as shown in FIG. 5. Whole stillage is collected from a fermentation process and separated into a wet cake and thin stillage. The thin stillage is further separated into a high protein stream and a low protein stream. One or more chemicals are added to the low protein stream to precipitate low protein components. For example, ammonia can be added to the low protein stream increasing its pH and decreasing the solubility of dissolved minerals, such as magnesium ammonium phosphate, commonly known as struvite. The minerals can be removed as a precipitate, thus increasing the protein content of the low protein stream. The low protein components can be removed by any suitable mechanisms including, but not limited to: quiescent decantation, centrifuge, filtration, membrane separation, or dissolved air flotation. The low protein stream can be concentrated prior to precipitation. The low protein stream is concentrated before adding to the wet cake. The wet cake can be dried before or after the addition of the low protein stream. Struvite can be sold as a valuable inorganic fertilizer. The digester effluent is now reduced in another non-protein component. The digester effluent with removed non-proteins is concentrated and added to wet cake to mitigate protein dilution effects in WDGS and DDGS.

It is common in the anaerobic digestion industry to remove magnesium-ammonium-phosphate (“struvite”) prior to or in parallel with digestion to prevent build-up of struvite deposits within the digester. Thus in another embodiment of the present invention, struvite removal is performed prior to or in parallel with anaerobic digestion.

In another embodiment of the present invention, membrane filtration can be used to remove the low protein components from the low protein stream. Whole stillage is collected from a fermentation process and separated into wet cake and thin stillage. The thin stillage is further separated into a high protein stream and a low protein stream. The low protein stream is directed through a filter of sufficient pore size to selectively remove the low protein components such as glycerol and lactic acid. Soluble proteins of a size established by the membrane pore size can be collected in the membrane concentrate (retentate). The filter can be of any suitable design including, but not limited to: hollow fiber, cross flow, and spiral wound. The treated low protein stream, now reduced in non-protein components and potentially enriched in soluble proteins, is added to the wet cake. The low protein stream can be concentrated prior to filtration. The treated low protein stream can be concentrated before adding to the wet cake. The wet cake can be dried before or after the addition of the low protein stream.

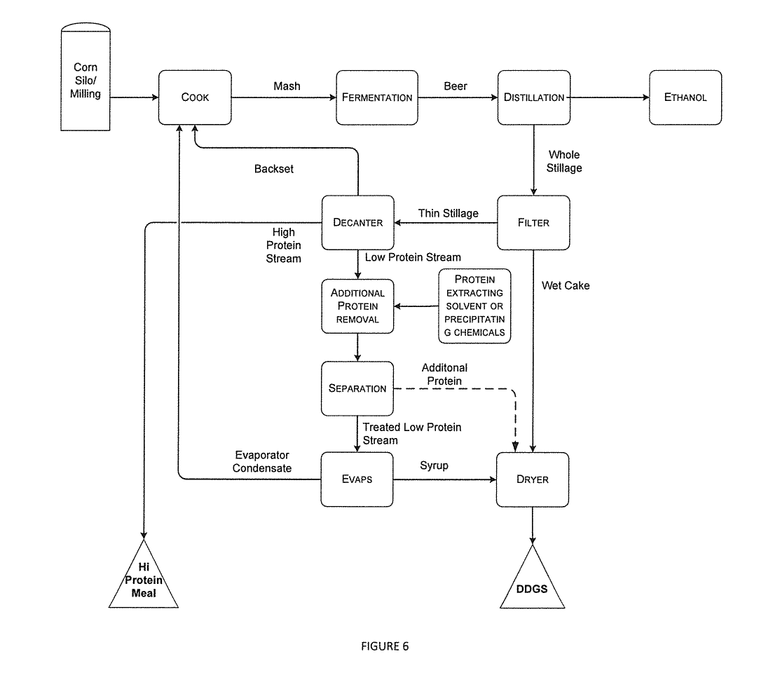

In another embodiment of the present invention, residual protein components in the low protein stream can be recovered as shown in FIG. 6. Whole stillage is collected from a fermentation process and separated into a wet cake and thin stillage. The thin stillage is further separated into a high protein stream and a low protein stream. Additional protein is recovered from the low protein stream. The additional protein recovered from the low protein stream is added to the wet cake. Additional protein can be recovered from the low protein stream by any suitable method. For example, proteins can be “salted out” by the addition of anionic or cationic salts to decrease the solubility of proteins in solutions. An organic solvent, such as ethanol, can be added, decreasing the dielectric constant of the solution and aids in denaturation of the protein therefore decreasing solubility. The pH of the solution can be adjusted to isoelectric points, which creates a net neutral charge on a protein, therefore decreasing interaction with the solution. Proteins can also be concentrated by the addition of polymeric agents that decrease the amount of free water for solvation. Protein can be separated and recovered by any suitable mechanism including, but not limited to, filtration, membrane filtration, centrifugation, dissolved air floatation or quiescent decantation. The low protein stream is concentrated before removal of protein. The recovered protein can be further concentrated prior to adding to wet cake. The wet cake can be dried before or after the addition of the protein.

Removal of Non-Protein Components from Wet Cake

In another method, protein levels in the wet cake are increased by removing non-protein components from the wet cake. For example, the fiber in the wet cake can be hydrolyzed. As previously described, fiber can be hydrolyzed by chemical and/or enzymatic means. The types of chemical and/or enzymatic treatments chosen allow for selective hydrolysis of hemicellulose or cellulose or both. Many schemes for chemical and/or enzyme assisted hydrolysis are conceivable to those skilled in the art. For example, hydrolysis of hemicellulose by dilute acid treatment can be followed step-wise by the enzymatic hydrolysis of cellulose (and residual hemicellulose) by a mixture of cellulases and hemicellulases. In another example, selective partial hydrolysis of cellulose is possible by addition of cellulose enzymes only as has been demonstrated by Kim et al. for DDGS. Hydrolysis can be sequential, for example, by separating hemicellulose hydrolysate from the partially digested solids and then hydrolyzing cellulose in a subsequent step. Any of the myriad chemical and enzymatic treatment schemes can be applied in the present invention to hydrolyze and remove fiber components from wet cake and thereby increase protein content of the residual cake. The hydrolysate sugars can be recovered and utilized by recycle to the upstream fermentation process from which wet cake is derived. Alternatively the hydrolysate sugars derived from the wet cake can be utilized in a fermentation process requiring a different organism producing the same or a different product than the fermentation process from which the source fiber was obtained.

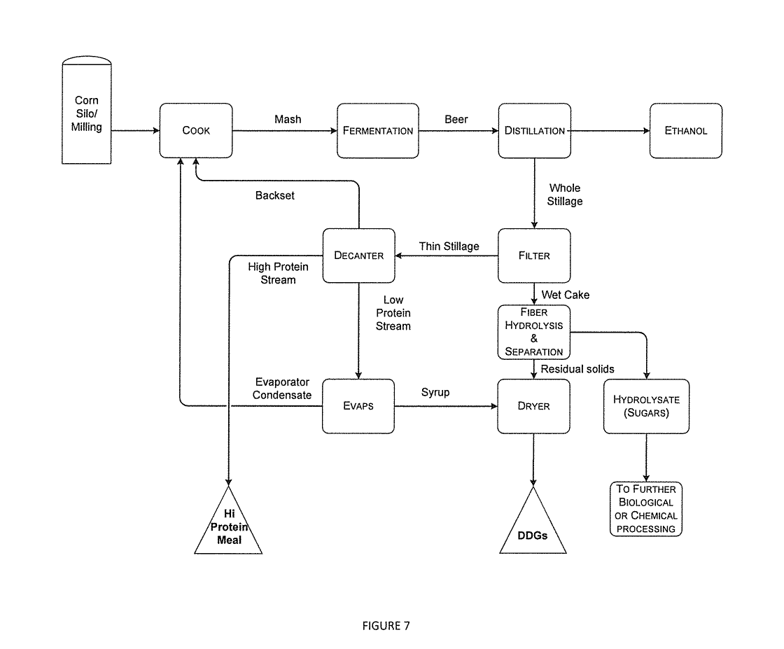

Thus in one embodiment of the present invention, as shown in FIG. 7, whole stillage is collected and separated into wet cake and thin stillage by for example filtration. Filtration can include a series of filter elements or devices. The filtered wet cake can be washed to improve protein transfer into the thin stillage stream. The thin stillage is separated into a high protein stream and a low protein stream by for example a centrifuge such as a decanting centrifuge or nozzle disc centrifuge. The wet cake from filtration is collected and subjected to a hydrolysis process to digest at least some of the fiber. The wet cake can further be ground and/or centrifuged prior to fiber hydrolysis. A liquid hydrolysate containing sugars from fiber hydrolysis is removed from the residual wet cake solids. The hydrolyzed wet cake can be washed to improve removal of sugars released from the hydrolysis process. The low protein stream is evaporated and added to the wet cake. The wet cake can be dried before or after the addition of the thin stillage, low protein stream, or any treated low protein stream, in this method or in any of the above methods.

The hydrolyzed sugars are subsequently converted to valuable products by biological or chemical processes. In one embodiment, wet cake hydrolysis is designed to selectively produce six-carbon sugars that can be fermented by the micro-organisms used in the upstream ethanol fermentation process from which the wet cake was produced.

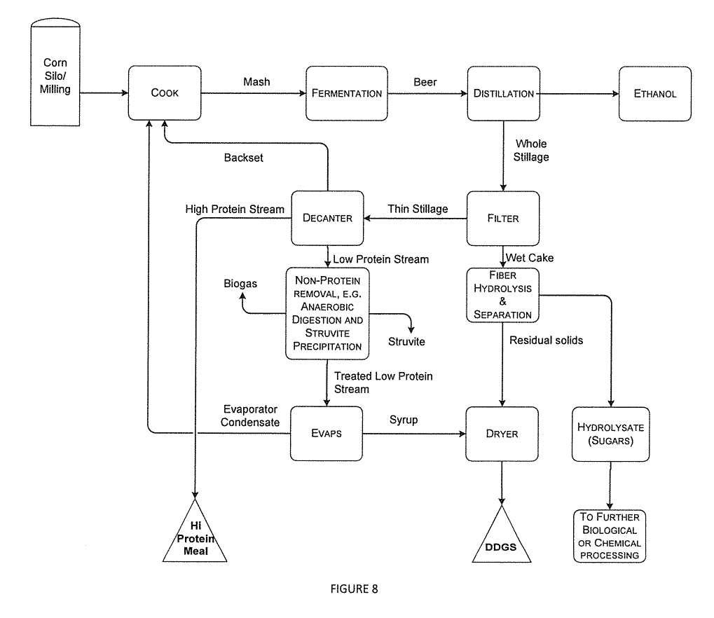

In another embodiment non-protein components are removed from both the low-protein stream and the wet cake. As shown in FIG. 8, whole stillage is separated into wet cake and thin stillage with a filter or other suitable device such as a decanter centrifuge. At least some of the wet cake can be hydrolyzed to give residual solids and a liquid hydrolysate containing hydrolysis sugars. Thin stillage can be separated by for example a centrifuge to produce a low protein stream and a high protein stream that can be subsequently dried to produce a high protein meal. Non-protein components can be removed from the low protein stream by for example anaerobic digestion and struvite precipitation. The treated low protein stream can be concentrated by evaporation and added to the residual solids to produce DDGS having an acceptable protein content. This is not intended to be a limiting embodiment of the invention and those skilled in the art will appreciate that other methods of removing non-protein components from the low protein stream and the wet cake can be used.

Therefore the present invention provides for a method of mitigating protein depletion in wet cake and DDGS in a grain fermentation process, while simultaneously producing a high protein distiller's grain co-product containing greater than 40 wt % protein on a dry matter basis.

The present invention provides for a method of producing a high protein distiller's grain co-product and simultaneously improving protein levels in wet cake and DDGS by separating whole stillage or spent grains into wet cake and thin stillage, separating at least some of the thin stillage into a high protein stream and a low protein stream, producing a high protein distiller's grain co-product from the high protein stream, treating the low protein stream to remove some or all of the non-protein components, and adding at least some of the treated low protein stream to at least some of the wet cake.

The present invention provides for a method of producing a high protein distiller's grain co-product and simultaneously improving protein levels in wet cake and DDGS by separating whole stillage or spent grains into wet cake and thin stillage, separating at least some of the thin stillage into a high protein stream and a low protein stream, producing a high protein distiller's grain co-product from the high protein stream, removing and recovering additional protein components from the low protein stream and adding the recovered protein to the wet cake.

The present invention provides for a method of producing a high protein distiller's grain co-product and simultaneously improving protein levels in wet cake and DDGS by separating whole stillage or spent grains into wet cake and thin stillage, separating at least some of the thin stillage into a high protein stream and a low protein stream, producing a high protein distiller's grain co-product from the high protein stream, removing some or all of the non-protein components from at least some of the wet cake, and adding at least some of the low protein stream to the wet cake.

The present invention provides for a method of managing the protein content of multiple distiller's grain co-products in a fermentation facility by treating a low-protein stream or wet cake or both to remove non-protein components and combining treated and untreated streams in proportions needed to achieve desired protein levels in multiple distiller's grain co-products.

The present invention also provides for a method of managing the protein content of multiple distiller's grain co-products of a fermentation process by separating whole stillage or spent grains into wet cake and thin stillage, removing a protein rich distiller's grain co-product having greater than 40 wt % protein on a dry matter basis from the thin stillage and producing a reduced protein thin stillage, and removing at least some non-protein components from the wet cake and producing a protein enriched wet cake. All or a portion of the reduced protein thin stillage can be added to at least some of the protein enriched wet cake, resulting in a mixture of reduced protein thin stillage and protein enriched wet cake. The mixture can be dried. The protein enriched wet cake can also be dried. At least some non-protein components can be removed from the reduced protein thin stillage prior to adding the reduced protein thin stillage to the protein enriched wet cake. This removal step can be performed by a hydrolysis process such as cellulose hydrolysis, hemi-cellulose hydrolysis, or combinations thereof. The method can further include the step of pretreating the wet cake prior to hydrolysis by a process such as protease treatment, lipase treatment, mechanical size reduction, acid treatment, alkali treatment, hydrothermal treatment, steam explosion, ammonia fiber expansion, ionic liquid extraction, or combinations thereof. Hydrolyzed wet cake can be separated into a liquid containing hydrolysis sugars and residual wet cake solids. The method can further include the step of converting the hydrolysis sugars to chemical products by a process such as a chemical process or a biological process. The hydrolysis sugars can be recycled to a step upstream of the fermentation process from whence the hydrolyzed wet cake was derived. Each of these steps is described in detail above.

The present invention provides for a method of managing protein by separating whole stillage or spent grains into wet cake and thin stillage, removing a protein rich distiller's grain co-product having greater than 40 wt % protein on a dry matter basis from the thin stillage and producing a reduced protein thin stillage, adding a micro-organism to the reduced protein thin stillage and growing biomass having higher protein content on a dry weight basis than wet cake, consuming non-protein components of the reduced protein thin stillage during the course of growing the biomass, and harvesting the biomass. At least a portion of the harvested biomass can be added to wet cake. The growing step can be anaerobic or aerobic. Essential nutrients can be added to the reduced protein thin stillage. The method can further include the step of dewatering during the harvesting step and producing a biomass having low free water content and an aqueous effluent. The biomass can be dried. The method can further include the step of adding at least a portion of the harvested biomass to at least a portion of the protein rich distiller's grain co-product, resulting in a mixture of harvested biomass and protein rich distiller's grain co-product. This mixture can then be dried. Each of these steps is described in detail above.

The present invention further provides for a system of managing the protein content of multiple distiller's grain co-products in a grain fermentation facility by separating whole stillage or spent grains into wet cake and thin stillage, removing a protein rich distiller's grain co-product having greater than 40 wt % protein on a dry matter basis from the thin stillage and producing a reduced protein thin stillage, removing non-protein components from a product chosen from the group consisting of the reduced protein thin stillage, the wet cake, and both the reduced protein thin stillage and the wet cake, and achieving a desired protein content in multiple distiller's grain co-products by combining protein containing streams chosen from the group consisting of thin stillage, wet cake, reduced protein thin stillage, reduced protein thin stillage having non-protein components removed, wet cake having non-protein components removed, protein rich distiller's grain co-product, biomass grown on reduced protein thin stillage, evaporated concentrates of substantially liquid streams in the preceding list, and dried forms of substantially wet solids in the preceding list.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

### Methods of Analysis

### Analytical Methods Common to Multiple Examples

The following analytical methods, shown in TABLE 1, established by AOAC International, were used throughout multiple examples. Other methods are described within specific examples.

TABLE 1

| Analysis | AOAC Method # |
|---|---|
| Dry Weight or Total | 934.01 (24 h, 105° C. method) |
| Solids (w/w) | |
| Crude Protein | 990.03 (Kjeldahl method) |

### Example 1

### Analysis of Low Protein Stream

Procedures

For the present EXAMPLE 1, whole stillage obtained from a commercial ethanol plant was filtered through a 600 micron pan filter. The filtrate and retentate were collected. The filtrate was heated to 250° F. and held at that temperature for 40 minutes, then cooled to 180° F. The filtrate was then centrifuged to separate the filtrate into a high protein stream and a low protein stream. The filtrate, retentate, high protein stream, and low protein stream were analyzed. The results are summarized in TABLE 2 on a theoretical 100 kilograms whole stillage dry solids basis.

TABLE 2

| | Stream Dry Solids | |
|---|---|---|
| | Weight based on | Protein |
| | 100 kg Whole | wt % |
| Product Stream | Stillage dry solids | (dry basis) |
| Whole Stillage | 100.0 | 25.90% |
| Wet Cake | 46.0 | 29.20% |
| Filtrate | 54.0 | 26.80% |
| High Protein Fraction | 31.05 | 41.50% |
| Low Protein Fraction | 22.95 | 11.30% |
| WDGS (=Wet Cake + Low Protein | 68.95 | 23.24% |
| Fraction) | | |

Results and Discussion

The separation of whole stillage into wet cake and thin stillage results in a wet cake with a protein level of 29.20%. WDGS are formed from the addition of the low protein stream to wet cake and hence the WDGS protein concentration is calculated to be 23.24%, i.e. 100%*[46(0.292)+22.95(0.113)]/(46+22.95). Removing some or all of the non-protein components of the low protein stream would increase the protein level and reduce the dry weight of the low protein stream. If, for example, the low protein stream was treated to remove 87.1% of the non-protein components (20.0 kg) the resultant protein levels and stream fractions would be as shown in TABLE 3. The total WDGS solids are reduced from 68.95% to 48.95% of the original whole stillage solids, but WDGS protein level is increased from 23.24% to 28.12%. This example shows that a high protein fraction having a protein content greater than 40 wt % on a dry basis can be isolated from whole stillage resulting in a protein depleted wet cake. By removing non-protein components from the low protein fraction of thin stillage, the protein content of the wet cake can be improved.

TABLE 3

| | Stream Dry Solids | |
|---|---|---|
| | Weight based on | Protein |
| | 100 kg Whole | wt % |
| Product Stream | Stillage dry solids | (dry basis) |
| Whole Stillage | 100.0 | 25.90% |
| Wet Cake | 46.0 | 29.20% |
| Filtrate | 54.0 | 26.80% |
| High Protein Fraction | 31.05 | 41.50% |
| Low Protein Fraction | 22.95 | 11.30% |
| Non-protein removed by treatment of | −20.0 | 0.0% |
| Low Protein fraction | | |
| Treated Low Protein Fraction | 2.95 | 11.30% |
| WDGS (=Wet Cake + Treated Low | 48.95 | 28.12% |
| Protein Fraction) | | |

### Example 2

### Acid Hydrolysis of Wet Cake to Increase Protein

Procedures

For the present EXAMPLE 2, whole stillage obtained from a commercial ethanol plant was filtered through a 600 micron pan filter. The filtrate and wet cake (retentate) were collected.

Control: Approximately 50 g of wet cake (retentate) having a total solids concentration of 20 wt % was mixed with deionized water to form a 10 wt % slurry and then centrifuged in 50 mL conical tubes. The supernatant was carefully decanted and the pelleted solids were washed by re-suspending the pellet in approximately 1 volume of deionized water, centrifuging and decanting the supernatant. Samples of first supernatant, final pellet, and wash water were collected and analyzed for solids and protein content.

Treatment 1: Approximately 50 g of wet cake (retentate) having a total solids concentration of 20 wt % was mixed with deionized water to form a 10 wt % solids slurry. The slurry was heated to 100° C., stirred for 1 hour and then centrifuged in 50 mL conical tubes. The supernatant was carefully decanted and the pelleted solids were washed by re-suspending the pellet in approximately 1 volume of deionized water, centrifuging and decanting the supernatant. Samples of first supernatant, final pellet, and wash water were collected and analyzed for solids and protein content.

Treatment 2: Approximately 50 g of wet cake (retentate) having a total solids concentration of 20 wt % was mixed with 0.5 M HCl (in deionized water) to form a 10 wt % solids slurry. The slurry was heated to 100° C., stirred for 1 hour and then centrifuged in 50 mL conical tubes. The supernatant was carefully decanted and the pelleted solids were washed by re-suspending the pellet in approximately 1 volume of deionized water, centrifuging and decanting the supernatant. Samples of first supernatant, final pellet, and wash water were collected and analyzed for solids and protein content.

Results and Discussion

The results of the experiment are shown in TABLE 4. Treatment 1 (hot water) increased the wet cake protein level, as compared to the control, from 29.4 dw % to 33.0 dw %, an increase of 32%. Treatment 2 (0.5 M HCl) increased the wet cake protein level, as compared to the control, from 29.4 dw % to 40.4 dw %, an increase of 39.4%. If the low protein stream from Example 1 is added to the treated wet cake of Example 2 to form WDGS, the calculated WDGS protein levels shown at the bottom of TABLE 4 can be obtained. The protein levels in the WDGS from wet cake treatment 1 and treatment 2 are increased from 19.65 dw % (control) to 25.18 dw % and 28.5 dw % respectively. This example shows that hot dilute acid hydrolysis is a very effective mechanism to hydrolyze non-protein components of wet cake and thereby increase the protein content of wet cake. Hot water treatment of wet cake also removes non-protein components but not as effectively as dilute acid. These treatments mitigate the protein dilution effect of adding, for example, low protein syrup (produced from reduced protein thin stillage) to wet cake.

TABLE 4

| | Treatment 1 | | Treatment 2 | |
|---|---|---|---|---|
| | (Hot Water) | | (0.5M HCl) | |
| | Dry Solids | | Dry Solids | |
| | Weight based | | Weight based | |
| | on 100 kg | Protein | on 100 kg | Protein |
| | Whole Stillage | (wt % dry | Whole Stillage | (wt % dry |
| Product Stream | dry solids | basis) | dry solids | basis) |
| Whole Stillage | 100.00 | 25.90% | 100.00 | 25.90% |
| Wet Cake | 46.00 | 29.20% | 46.00 | 29.20% |
| Filtrate | 54.00 | 26.80% | 54.00 | 26.80% |
| High Protein | 31.05 | 41.50% | 31.05 | 41.50% |
| Fraction | | | | |
| Low Protein | 22.95 | 11.30% | 22.95 | 11.30% |
| Fraction | | | | |
| Non-protein | −5.29 | 0.00% | −12.75 | 0.00% |
| removed by | | | | |
| treatment of | | | | |
| Wet Cake | | | | |
| Treated Wet | 40.70 | 33.00% | 33.25 | 40.4% |
| Cake Fraction | | | | |
| WDGS (=Wet | 63.65 | 25.18% | 56.20 | 28.15% |
| Cake + Treated | | | | |
| Low Protein | | | | |
| Fraction) | | | | |

### Example 3

### Enzymatic Hydrolysis of Wet Cake to Increase Protein

In a proof of concept experiment, samples of wet cake having reduced protein content were prepared by the mechanical separation methods described below and then subjected to hydrolysis with cellulase enzymes only (no hemi-cellulase). Prior to hydrolysis, the wet cake was not subjected to any of the pretreatments common to cellulosic ethanol industry. In this embodiment, the hydrolysate is primarily comprised of glucose and is thus suitable for recycle to the same fermentation process from whence the wet cake was produced.

Supplemental Analytical Methods

Glucan composition of the wet cake (retentate) prior to hydrolysis was determined as glucose resulting from the NREL two-stage acid digestion method for structural carbohydrates. Reference publication: Sluiter et al., Determination of Structural Carbohydrates and Lignin in Biomass. Laboratory Analytical Procedure (LAP). National Renewable Energy Laboratory. Technical Report: NREL/TP-510-42618. Issued Date: 25 Apr. 2008.

Hydrolysis sugars (glucose, xylose, arabinose) in centrifuged hydrolysate samples were determined by HPLC with these conditions: Column: Biorad Aminex HPX-87H; Guard column: Cation H; Flow rate: 0.6 mL/min, Mobile phase: 5 mM sulfuric acid, Column oven temperature: 50° C., Detector: Refractive Index.

Procedures

Preparation of wet cake. Whole stillage obtained from a commercial ethanol plant was used to prepare the wet cake samples identified as “unground retentate” and “ground retentate” according to the following procedures. The grinding process liberates more protein from the wet cake fiber as manifest by the crude protein analysis in Table 7.

Unground Retentate (Sample “UG3C”): About 72% of the Unground Retentate sample was produced by simply filtering whole stillage through a 200 micron screen and collecting the filtered solids (retentate). The balance of the Unground Retentate sample was subjected to a wash step by suspending the filtered solids in hot deionized water (180° F.) water and filtering again through 200 micron screen. The unwashed material (72%) and washed material (28%) were combined and uniformly mixed to produce a homogeneous sample of Unground Retentate.

Ground Retentate (Sample “G2C”): 500 ml aliquots of whole stillage were ground in a Ninja Master Prep Blender (500 ml container) for 5 minutes total grind time (on 30 seconds off 30 seconds repeated to avoid overheating the blender). The ground whole stillage was filtered through a 200 micron screen. The material collected on the screen (retentate) was washed by suspending in 300 ml of 180° F. (82° C.) deionized water and filtering again through the 200 micron screen. Multiple 500 mL aliquots of whole stillage were processed through the grinding, filtration and wash process to obtain the final sample of Ground Retentate.

Hydrolysis of wet cake: The Ground and Unground Retentate wet cake samples were analyzed for moisture and glucan content. Per Table 6 below, wet cake was added to a 250 mL shake flasks to achieve 100 mL total hydrolysis volume at 2.5% glucan loading. Novozymes Ctec2 cellulase was added at 15 mg enzyme/g glucan and distilled water was added to bring the reaction volume to 100 mL. Chloramphenicol (100 μL) and 10M KOH (60 or 120 μL) were added for bacterial inhibition and pH adjustment respectively. The hydrolysis reaction was allowed to proceed for 24 hours at 50 degrees C. with shaking at 250 rpm on an orbital shake table. After 24 hours hydrolysis, the reaction mass was centrifuged to separate hydrolysate liquid (supernatant) from residual solids (pellet) and the supernatant was analyzed for sugars.

TABLE 6

| Wet Cake Sample ID | G2C | UG3C |
|---|---|---|
| Target Glucan loading | 2.50% | 2.50% |
| Target Reaction Volume (mL) | 100 | 100 |
| Wet Cake Moisture content (%) | 84.30% | 81.10% |
| Target enzyme loading (mg protein/g glucan) | 15 | 15 |
| Biomass Glucan content (wt % dry basis) | 18.12% | 17.92% |
| Biomass addition to flask (g wet wt) | 87.88 | 73.81 |
| Biomass dry wt calculated (g) | 13.80 | 13.95 |
| Glucan dry wt calculated (g) | 2.50 | 2.50 |
| Biomass solids concentration in flask (wt %) | 13.81% | 13.96% |
| Chloramphenicol addition to flask (μL) | 100 | 100 |
| Distilled water addition to flask (mL) | 11.75 | 25.75 |
| Ctec2 solution (174 mg/mL) addition to flask, | 215.89 | 215.89 |
| (μL) | | |
| 10M KOH addition to flask (μL) | 60.00 | 120.00 |
| measured pH at 1.5 hr | 4.45 | 4.34 |

Results and Discussion

The concentrations of sugars in the hydrolysate were determined by HPLC and then calculated and presented on a total reaction volume basis (100.0 mL) in Table 7. The total production of sugar is also calculated and shown in Table 7.

TABLE 7

| Sample ID | G2C | UG3C |
|---|---|---|
| Glucose concentration in reaction volume (g/L) | 7.85 | 10.39 |
| Xylose concentration in reaction volume (g/L) | 0.51 | 0.31 |
| Galactose concentration in reaction volume (g/L) | 0.15 | 0.30 |
| Arabinose concentration in reaction volume (g/L) | 0.75 | 0.44 |
| Glucose total production (g) | 0.78 | 1.04 |
| Xylose total production (g) | 0.05 | 0.03 |
| Galactose total production (g) | 0.01 | 0.03 |
| Arabinose total production (g) | 0.08 | 0.04 |
| Glucan conversion (%) | 31.4% | 41.6% |
| Crude protein content, prior to hydrolysis, wt % dry | 25.80 | 29.65 |
| basis | | |
| Total Hydrolysate sugars removed, calculated (g) | 0.93 | 1.14 |
| Biomass solids remaining after hydrolysis, calculated | 12.87 | 12.81 |
| (g) | | |
| Protein content after hydrolysis, calculated (wt % dry | 27.5% | 32.3% |
| basis) | | |

As expected for cellulase-only hydrolysis, the production of the cellulose monomer glucose was much higher than the production of xylose, galactose or arabinose, monomeric sugars that comprise hemi-cellulose. The total glucan conversion was 31.4 and 41.6% for the ground and unground retentate (wet cake) respectively. Assuming that the hydrolysis sugars are completely removed from the residual hydrolysis solids by filtering and washing, the calculated protein content of the treated wet cake increases by approximately 2 percentage points in both cases. This example provides further evidence that the present invention serves to increase the protein content of wet cake and mitigate the protein dilution effect that would occur if low protein thin stillage syrup was added to wet cake to produce WDGS or DDGS. This example should not be interpreted as limiting and parameters such as type and degree of cellulose/hemi-cellulose pretreatment, enzyme types, source and loading, hydrolysis time and temperature, biomass loading and reaction hydrodynamics/mixing are parameters which can be optimized for maximum yield.

### Example 4

### Anaerobic Digestion of Reduced Protein Thin Stillage

Procedures

For the present EXAMPLE 4, whole stillage obtained from a commercial ethanol plant was filtered through a 600 micron pan filter. The filtrate and retentate were collected. The filtrate was heated to 250° F. and held at that temperature for 40 minutes, and then cooled to 180° F. The filtrate was then centrifuged to separate the filtrate into a high protein stream and a low protein stream. The pH adjusted low protein stream with nutrients was fed to a 15 L Up-Flow Anaerobic Sludge bed reactor (UASB) seeded with bacteria obtained from an operating anaerobic digester (also known as a “methanator”) of a commercial ethanol plant and used for treatment of evaporator condensate. The bacterial consortium consists of second phase anaerobic digestion organisms, primarily acetogens and methanogens. The reactor was run with a hydraulic residence time (HRT) of 12 hours and a bed solids content of 6%. Samples of the low protein stream were collected before feeding the reactor and the effluent was sampled after passing out of the reactor system. Feed and effluent samples were analyzed by HPLC for total solids, glycerol, organic acids and protein content.

Results and Discussion

TABLE 8 shows the analysis of the low protein feed stream and effluent from the anaerobic digester system at steady state.

TABLE 8

| COMPONENT | MEASURED CHANGE |
|---|---|
| Glycerol | 95% Degradation |
| Organic Acids | 75% Degradation |
| Protein | Feed: 11% |
| (wt % on dry basis) | Effluent: 19% |

Organic acids and glycerol were effectively degraded by the anaerobic bacteria to create biogas. When those components were removed from the process stream, the concentration of protein increased in the low protein stream from 11% to 19% (dry basis). In a typical ethanol plant the low protein stream is evaporated and the syrup is added to the wet cake to produce DDGS. When the 11% low protein stream is added to wet cake that contains 28.6% protein the resulting material decreases in protein content to 23.5%. When the 19% AD low protein process stream is added to the wet cake the protein content increases to 26.7% protein. This example provides further evidence that the present invention serves to increase the protein content of the low protein stream and hence WDG and DDGS.

What is claimed is:

1. A method of managing the protein content of multiple distiller's grain co-products of a fermentation process, including the steps of:
separating whole stillage or spent grains into wet cake and thin stillage;
removing a protein rich distiller's grain co-product having greater than 40 wt % protein on a dry matter basis from the thin stillage and producing a reduced protein thin stillage;
removing at least some non-protein components from the reduced protein thin stillage; and
adding all or a portion of the reduced protein thin stillage, having at least some non-protein components removed, to at least some of the wet cake.

2. The method of claim 1, wherein said separating step is chosen from the group consisting of decanting centrifuge, disc stack centrifuge, nozzle disc centrifuge, filtering centrifuge, pressure screens, gravity screens, paddle screen, static screen, vibratory screen and combinations thereof.

3. The method of claim 1, wherein said step of removing a protein rich distiller's grain co-product is accomplished with a mechanism chosen from the group consisting of adding one or more protein agglomerating chemicals, filtration, membrane filtration, dissolved air floatation, quiescent decantation, decanting centrifuge, disc stack centrifuge, nozzle disc centrifuge, filtering centrifuge, paddle screen, static screen, vibratory screen, and combinations thereof.

4. The method of claim 1, wherein said step of removing at least some non-protein components is accomplished with a mechanism chosen from the group consisting of anaerobic digestion, ion exchange, struvite precipitation, microfiltration membranes, ultrafiltration membranes, nanofiltration membranes, reverse osmosis membranes, and combinations thereof.

5. The method of claim 1, wherein said step of removing of non-protein components from is performed at a time consisting of before concentrating the reduced protein thin stillage and after concentrating the reduced protein thin stillage.

6. The method of claim 5, wherein said concentrating step is performed with a multi-effect evaporator.

7. The method of claim 1, further including, after said adding step, the step of drying the wet cake.

8. The method of claim 1, further including, removing additional protein from the reduced protein thin stillage.

9. The method of claim 8, further including, adding the additional protein to wet cake.

10. A method of managing the protein content of multiple distiller's grain co-products of a fermentation process, including the steps of:
separating whole stillage or spent grains into wet cake and thin stillage;
removing a protein rich distiller's grain co-product having greater than 40 wt % protein on a dry matter basis from the thin stillage and producing a reduced protein thin stillage; and
removing at least some non-protein components from the wet cake and producing a protein enriched wet cake.

11. The method of claim 10, wherein all or a portion of the reduced protein thin stillage is added to at least some of the protein enriched wet cake, resulting in a mixture of reduced protein thin stillage and protein enriched wet cake.

12. The method of claim 11, further including the step of drying the mixture.

13. The method of claim 10, further including the step of drying the protein enriched wet cake.

14. The method of claim 10, further including the step of removing at least some non-protein components from the reduced protein thin stillage prior to adding the reduced protein thin stillage to the protein enriched wet cake.

15. The method of claim 10, wherein said removing at least some non-protein components step is performed by a hydrolysis process chosen from the group consisting of cellulose hydrolysis, hemi-cellulose hydrolysis, and combinations thereof.

16. The method of claim 11, further including the step of pretreating the wet cake prior to hydrolysis by a process chosen from the group consisting of protease treatment, lipase treatment, mechanical size reduction, acid treatment, alkali treatment, hydrothermal treatment, steam explosion, ammonia fiber expansion, ionic liquid extraction, and combinations thereof.

17. The method of claim 11, further including the step of separating hydrolyzed wet cake into a liquid containing hydrolysis sugars and residual wet cake solids.

18. The method of claim 13, further including the step of converting the hydrolysis sugars to chemical products by a process chosen from the group consisting of a chemical and a biological process.

19. The method of claim 13, further including the step of recycling the hydrolysis sugars to a step upstream of the fermentation process from whence the hydrolyzed wet cake was derived.

20. A method of managing protein, including the steps of:
separating whole stillage or spent grains into wet cake and thin stillage;
removing a protein rich distiller's grain co-product having greater than 40 wt % protein on a dry matter basis from the thin stillage and producing a reduced protein thin stillage;
adding a micro-organism to the reduced protein thin stillage and growing biomass having higher protein content on a dry weight basis than wet cake;
consuming at least some of the non-protein components of the reduced protein thin stillage during the course of growing the biomass; and
harvesting the biomass.

21. The method of claim 20, further including the step of adding at least a portion of the harvested biomass to wet cake.

22. The method of claim 20, wherein said growing step is chosen from the group including anaerobic or aerobic.

23. The method of claim 20, further including the step of adding essential nutrients to the reduced protein thin stillage

24. The method of claim 20, further including the step of dewatering during said harvesting step and producing a biomass having low free water content and an aqueous effluent.

25. The method of claim 24, further including the step of drying the biomass.

26. The method of claim 20 further including the step of adding at least a portion of the harvested biomass to at least a portion of the protein rich distiller's grain co-product, resulting in a mixture of harvested biomass and protein rich distiller's grain co-product.

27. The method of claim 22, further including the step of drying the mixture.

28. A system of managing the protein content of multiple distiller's grain co-products in a grain fermentation facility including the steps of
separating whole stillage or spent grains into wet cake and thin stillage;
removing a protein rich distiller's grain co-product having greater than 40 wt % protein on a dry matter basis from the thin stillage and producing a reduced protein thin stillage;
removing non-protein components from a group consisting of the reduced protein thin stillage, the wet cake, and both the reduced protein thin stillage and the wet cake; and
achieving a desired protein content in multiple distiller's grain co-products by combining, proportionately and as needed, protein containing streams chosen from the group consisting of thin stillage, wet cake, reduced protein thin stillage, reduced protein thin stillage having non-protein components removed, additional protein removed from reduced protein thin stillage, wet cake having non-protein components removed, protein rich distiller's grain co-product, biomass grown on reduced protein thin stillage, evaporated concentrates of substantially liquid streams in the preceding list and dried forms of substantially wet solids in the preceding list.
